(19) United States
(12) Patent Application Publication
(10) Pub. No.: US 2024/0016904 A1
(43) Pub. Date: Jan. 18, 2024

(54) METHODS OF TREATING SUPERFICIAL DIGITAL FLEXOR TENDON INJURIES, DEEP AND SUPERFICIAL MUSCLE/TENDON CONTRACTURE, NAVICULAR SYNDROME, AND SUSPENSORY LIGAMENT INFLAMMATION AND INJURIES IN QUADRUPED ANIMALS

(71) Applicant: VetMed Therapeutics, Inc., Gulf Breeze, FL (US)

(72) Inventors: J. Ben Renfroe, Gulf Breeze, FL (US); Daniel W. Carter, Gulf Breeze, FL (US)

(21) Appl. No.: 18/254,582

(22) PCT Filed: Nov. 24, 2021
(86) PCT No.: PCT/US2021/060807

§ 371 Date: May 25, 2023

Related U.S. Application Data
(60) Provisional application No. 63/118,575, filed on Nov. 25, 2020.

(51) Int. Cl. A61K 38/48; A61K 9/00; A61K 45/06; A61P 19/04

(52) U.S. Cl. CPC A61K 38/4893; A61K 9/0019; A61K 45/06; A61P 19/04

(57) ABSTRACT

Methods of treating superficial digital flexor tendon injuries, deep and superficial muscle/tendon contracture, navicular syndrome, and suspensory ligament inflammation and injuries in the limb of a quadruped animal, such as hooved animals and other quadrupeds. Such abnormalities may be inflammatory or associated with pain. The abnormalities may be chronic or acute. Such abnormalities may involve anatomical structures a limb, neck, torso, and/or pelvis of the quadruped animal.

### FIELD OF THE INVENTION

The invention relates to the treatment of superficial digital flexor tendon injuries, deep and superficial muscle/tendon contracture, navicular syndrome, and suspensory ligament inflammation and injuries in quadruped animals with botulinum toxin (all types). More particularly, the method is effective in treating superficial digital flexor tendon injuries, deep and superficial muscle/tendon contracture, navicular syndrome, and suspensory ligament inflammation and injuries in the limb of a quadruped animal, such as hooved animals and other quadrupeds.

### BACKGROUND OF THE INVENTION

Tendon and ligament injuries of the limbs are common in all types of horses. Tendons are soft tissue structures that transfer the forces generated by muscle contraction into skeletal movement, provide skeletal support, and aid locomotion by storage and release of elastic energy. Although similar in ultra-structure, ligaments do not have a muscle component but instead provide alignment and stabilization of adjacent bones and passive resistance to motion. Kasashimi et al. suggests tendon and ligament injuries are the most common soft tissue injuries of Japanese thoroughbreds.

Superficial Digital Flexor Tendon Injuries:

The superficial flexor tendon is believed to function at its biomechanical limits in performance horses. The superficial digital flexor tendon is the most frequently injured soft tissue of the fore limb and it is estimated to be the leading cause of thoroughbred retirements from racing and 13% of national hunt horses suffered a superficial digital flexor tendon injury over a 1-year period.

Tendons are composed of sparse, metabolically active fibroblasts embedded in a highly organized extracellular matrix (amorphous ground substance and fibrous elements). Tendon injury may result from its poor regenerative properties following repetitive loading (strain) and accumulated degeneration of the collagen fibers and collagen-rich extracellular matrix. An inability of the cellular components to maintain and repair the extracellular matrix leads to intrinsic damage and failure. In addition, acute tendon failure may occur when exercise overload exceeds the structural strength of the tendon.

Healing can be divided into 3 stages (inflammation, reparative, and remodeling) although these processes are continuous and overlap. The inflammatory phase functions to remove damaged tissue and lasts a few days, overlapping with the reparative stage and deposition of tissue that will form the scar. The scar tissue then proceeds through a maturation phase where the new collagen type III transforms into collagen type I. The final scar is often larger than the original tendon and less flexible. Initial treatment of superficial digital flexor tendon injury is aimed at control of the inflammatory phase to limit the extent of tendon damage. Treatment has changed little over the previous decades and includes non-steroidal anti-inflammatory medications, (flunixin, phenylbutazone), cold therapy (cold water, ice bath), bandaging to control edema, stall confinement, pasture rest, and farrier intervention.

Adjunct treatments currently available include intra-lesional injection of platelet rich plasma, insulin-like growth factor, and mesenchymal stem cells. Current surgical therapies have questionable value and include tendon splitting (longitudinal incision) to drain the lesion, which may limit lesion size. Desmotomy of the accessory ligament (superior check ligament) of the superficial digital flexor tendon has been reported. One report has suggested that horses managed by superior check desmotomy (surgical transection) were 1.3 times more likely to complete five or more races than non-surgically managed horses, but were 1.2 times as likely to develop recurrent or new injuries after returning to training. Horses treated surgically were 5.5 times more likely to develop suspensory ligament desmitis than those managed non-surgically.

Deep and Superficial Muscle/Tendon Contracture:

Musculo-skeletal abnormalities are common in horses. The conditions can be congenital or acquired. Included in these abnormalities are flexural abnormalities that refer to the inability to fully extend a limb, as well as hyperextension due to the complex impact of forces across multiple joints in the leg. A common factor in some flexural deformities is excessive tensile forces exerted by the associated muscle on the deep digital flexor tendon or the superficial flexor tendon. Contraction of the deep digital flexor (DDF) or superficial digital flexor (SDF) is an acquired condition that can affect any equine limb, but most commonly affects one or both forelimbs. Untreated contraction of the digital flexors may result in remodeling of adjacent soft tissues and bone. These changes may result in permanent flexural abnormalities of form and function in the affected limb, limiting the use and economic value of the animal.

The treatment of neonatal flexural deformities depends upon severity of the abnormality and may resolve with bandaging, splints, and casts. Foals unable to extend the limb sufficiently to stand and nurse are often humanely destroyed because of poor prognosis. Other current interventions include medical and nutritional management, surgical intervention, and farrier care. Medical management attempts to control factors contributing to the positive feedback cycle of pain and contraction; restriction of exercise to reduce concussion of growing bone and cartilage; balanced nutrition to normalize bone and joint development; and the judicious use of anti-inflammatory medication to reduce pain. Cases that do not show significant improvement may require desmotomy of the accessory ligament of the deep digital flexor tendon (inferior check ligament). This results in a mild functional lengthening of the deep digital flexor tendon. Intervention at the superior check ligament results in similar changes in the superior digital flexor tendon.

Limited information is available on the long-term effects of accessory ligament desmotomy on the performance horse. Reports indicate that animals subjected to the procedure are able to perform, but data is not available regarding successful performance. This may impact the economic value of the horse.

Navicular Syndrome.

Navicular syndrome of horses is a lameness syndrome that is commonly associated with degeneration of the distal sesamoid bone (navicular bone). An additional descriptive clinical term is caudal heel pain syndrome. Navicular syndrome may present with a variety of clinical manifestations; therefore, it is unlikely to have a single etiology but rather pain resulting from any one or more of the structures located within the navicular or heel region of the digit that includes the navicular bone (distal sesamoid bone) and a complex of elastic and tendinous attachments (suspensory ligaments of the sesamoid, sesamoid impar ligament, collateral ligaments, T ligament, navicular bursa and deep digital flexor tendon). It is frequently associated with chronic, slowly progressive lameness of one or both fore or hind limbs, although acute onset may occur in fore or hind limbs.

Treatment of lame horses diagnosed with navicular syndrome include intra-articular corticosteroid injections, systemic non-steroid drugs, a multitude of stem-cell products, shock wave therapy, acupuncture, chiropractic procedures, farrier intervention and appliances of the hoof capsule and tiludronate. Despite the plethora of treatment options, the prognosis for recovery is guarded and, in chronic cases, is poor. Unfortunately, the standard treatment for navicular syndrome of horses is surgical transection (neurectomy) of the palmar nerves at the level of the pastern. This procedure is often complicated by the nature of the distal limb of the horse, that is, constant motion and adjacent to soil and manure resulting in frequent suture infection, wound dehiscence, and development of painful neuromas. Tiludronate is FDA approved for the treatment of navicular syndrome in horses, but has not proved effective because the evidence suggests: navicular bone may not be the inciting cause of disease; that the navicular bone changes noted on radiographs are not amendable to the effects of bis-phosphonates; non-steroidal anti-inflammatory drugs are poorly effective especially in chronic cases of navicular syndrome. Recent evidence has shown presence of inflammation within the region of the distal sesamoidean impar ligament attachment to the deep digital flexor tendon of horses diagnosed with navicular syndrome. However, horses with chronic navicular syndrome usually remain lame.

Suspensory Ligament Inflammation and Injuries.

Suspensory desmitis is damage and inflammation of the ligament, or its branches, caused by excessive loading forces, along with fatigue, of the limb muscles during intense exercise. Additionally, degenerative disorders of collagen and pituitary pars intermedia hyperplasia may exacerbate desmitis.

Treatment of horses whose lameness is because of proximal suspensory desmitis (PSD) of the hind limbs include prolonged confinement followed by a regimen of controlled, gradually increasing exercise. However, horses with PSD of one or both hind limbs usually remain lame. In one study, confinement followed by a regime of controlled, gradually increasing exercise enabled only 14% of horses with PSD of one or both hind limbs to return to their previous level of activity, without recurrent lameness, for more than a year.

Accordingly, there is a need for a proactive treatment that effectively treats or prevents superficial digital flexor tendon injuries, deep and superficial muscle/tendon contracture, navicular syndrome, and suspensory ligament inflammation and injuries. The present invention addresses such a need.

### SUMMARY OF THE INVENTION

The present invention relates to methods of treating superficial digital flexor tendon injuries, deep and superficial muscle/tendon contracture, navicular syndrome, and suspensory ligament inflammation and injuries of hooved animals and domesticated quadrupeds in need thereof by administering a botulinum toxin composition to one or more area of injection. The botulinum toxin exerts localized effects whether of muscle tissue or other tissues. This effect thereby reduces forces on the injured or painful tissue, allowing time for healing and/or other interventions.

Embodiments of the present disclosure can include a method for treating superficial digital flexor tendon injuries, deep and superficial muscle/tendon contracture, navicular syndrome, and suspensory ligament inflammation and injuries of a quadruped animal comprising: injecting a botulinum toxin, in a therapeutically effective amount, into one or more tissues or muscles of the animal. The Botulinum Toxin can be injected into at least one muscle that imparts torsional forces on one or more skeletal bones of the limb. Such muscles can include one or more muscles or ligaments of the limb are selected from deep digital flexor muscle, the superficial digital flexor muscle or the suspensory ligament.

Animals to be treated by the methods described herein can be a horse or camel and particularly a filly or a colt or a horse or camel less than 3 years old or less than a year old. In other embodiments, the horse or camel is an adult or greater than 3 year olds. In embodiments, the camel's or horse's injury is caused by stress caused by racing or training for races.

The abnormalities or injuries to be treated by the methods described herein can be acquired or congenital.

### DESCRIPTION OF FIGURES

FIG. 1 is a schematic of the anatomy of a lower portion of the horse leg and hoof with the pathological area of superficial digital flexor tendon shown in the shaded rectangular area.

FIG. 2 is a schematic of the anatomy of a lower portion of the horse leg and hoof with the pathological area of deep and superficial muscle/tendon contracture shown by the arrow.

FIG. 3 is a schematic of the anatomy of a hoof showing where the navicular bone is located;

FIG. 4 is a schematic of the anatomy of a lower portion of the horse leg and hoof with showing the suspensory ligament in need of treatment.

### DETAILED DESCRIPTION OF THE INVENTION

The methods of the invention are directed to treating or reducing the superficial digital flexor tendon injuries, deep and superficial muscle/tendon contracture, navicular syndrome, and suspensory ligament inflammation and injuries in quadrupeds, particularly those that are hooved or domesticated. These abnormalities or injuries are a common congenital or acquired malady affecting quadrupeds, particularly larger, long-legged animals, such as, without limitation, horses, zebras, giraffes, and camels.

While not wishing to be bound by a particular theory, abnormal and unbalanced muscle tone in specific muscle groups of the leg and shoulder may contribute to superficial digital flexor tendon injuries, deep and superficial muscle/tendon contracture, navicular syndrome, and suspensory ligament inflammation and injuries. As such, intervention at the muscular and myoneural level through the administration of botulinum toxin can result in an improvement or stabilization of such abnormalities/injuries.

While not wishing to be bound by a particular theory, it is believed that muscles act in an elastic manner, damping and modifying the spiral loading of the limbs' boney column during the protraction, stance or retraction phase of stride. Abnormal or unbalanced tone may exert excessive torsional forces on the skeleton, thereby causing or contributing to abnormalities or injuries such as increased hoof and pastern angles or posterior displacement of the knee. By decreasing the tension in one or more muscles, the excessive torsional forces can be alleviated, thereby reducing the degree or severity of the abnormalities.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve reducing the degree of the deformity or injury or alleviating one or more symptoms of the deformity or injury, although not necessarily all of the symptoms, of the deformity or injury, or attenuating the progression of the deformity or injury. Eliminating the deformity or injury from the animal is an optimal outcome of the practice of the methods of the invention. Embodiments of the invention include administering compositions of the invention to prevent or reduce the incidence or severity of the superficial digital flexor tendon injuries, deep and superficial muscle/tendon contracture, navicular syndrome, and suspensory ligament inflammation and injuries.

A “quadruped animal” to be treated according to the present methods can refer to, e.g., Equidae (e.g., horse, ass, zebra), Bovidae (e.g., cattle, bison, sheep, goat, yak, impala, antelope, hartebeest, wildebeest, gnu, gazelle, water buffalo, duiker), Cervidae (e.g., deer, elk, moose, reindeer, pudu, bororo, brocket, guemal, muntjac), Suidae (e.g., pig, hog, boar), Canidae (domesticated dog, wolf, fox, coyote, jackel), Felidae (e.g., domesticated cat, cheetah, ocelot, lynx, bobcat, mountain lion, leopard, puma, lion, jaguar, tiger), Rodentia (e.g., mouse, rat, guinea pig, chinchilla, agouti, porcupine, beaver, gopher), Lagomorpha (e.g., rabbit, jackrabbit, hare, pika), Camelidae (e.g., camel, llama, alpaca, guanaco, vicugna), Ursidae (e.g., bear, panda), Procyonidae (e.g., raccoon, coati, olingo), Mustelidae (polecat, weasel, ferret, mink, fisher, badger, otter, wolverine, marten, sable, ermine), Elephantidae (e.g., elephant), rhinoceros, and hippopotamus. In some embodiments, the quadruped animal is a domestic or commercially used animal. In some embodiments, the quadruped animal is a horse.

The quadruped animal to be treated according to the present methods can be an immature animal or an animal still undergoing musculoskeletal growth. In some embodiments, the quadruped animal can be less than 3 years, 2 years, 1 year, 6 months, 5 months, 4, months, 3 months, 2 months, or 1 month of age. In some embodiments, the quadruped animal is a filly or a colt. In some embodiments, the quadruped animal is a foal. In other embodiments, the animal is a mature animal, greater than 3 or 4 years old.

According to the invention, a method of treating superficial digital flexor tendon injuries, deep and superficial muscle/tendon contracture, navicular syndrome, and suspensory ligament inflammation and injuries in a quadruped animal comprises administering, such as by injection or intramuscular injection, an effective amount or a therapeutically effective amount of botulinum toxin to one or more target muscles in a limb (forelimb or hind limb) or shoulder of the animal. The treatment methods are directed to ameliorating, preventing, inhibiting, reversing, attenuating, alleviating, abrogating, minimizing, suppressing, reducing, decreasing, diminishing, stabilizing, eradicating, curing, or eliminating superficial digital flexor tendon injuries, deep and superficial muscle/tendon contracture, navicular syndrome, and suspensory ligament inflammation and injuries, and/or their associated symptoms caused thereby. The target muscle(s) within the limb or shoulder can vary depending on the nature of the deformity or injury.

In the case of an animal with superficial digital flexor tendon injuries, deep and superficial muscle/tendon contracture, navicular syndrome, and suspensory ligament inflammation and injuries, at least one target tissue can be one that imparts superficial digital flexor tendon injuries, deep and superficial muscle/tendon contracture, navicular syndrome, and suspensory ligament inflammation and injuries. The target muscle(s) within the limb or shoulder can vary depending on the nature of the musculoskeletal deformity.

To identify the target tissue, a clinician can physically examine the quadruped animal to identify areas of pathology. For example, the clinician can conduct a tactile examination to identify the superficial digital flexor tendon injuries, deep and superficial muscle/tendon contracture, navicular syndrome, and suspensory ligament inflammation and injuries. A tactile examination, electromyography and/or ultrasound may also be used for identification of muscles with increased tone.

Flexural Abnormalities

Flexural abnormalities or deformities refer to the inability to fully extend a limb, as well as hyperextension due to the complex impact of forces across multiple joints in the leg. A common factor in some flexural deformities is excessive tensile forces exerted by the associated muscle on the deep digital flexor tendon or the superficial flexor tendon. Contraction of the deep digital flexor (DDF) or superficial digital flexor (SDF) is an acquired condition that can affect any quadruped limb, particularly equine, but most commonly affects one or both forelimbs. Untreated contraction of the digital flexors may result in remodeling of adjacent soft tissues and bone. These changes may result in permanent flexural abnormalities of form and function in the affected limb, limiting the use and economic value of the animal. A flexural injury can present as a flexural abnormality and therefore can be treated in a similar fashion.

Increased tone of the deep and superficial digital flexor muscles can result in excess posterior pull on the distal phalanges. This pull is transmitted over multiple joints and results in a posterior displacement at the knee, increased pastern angle, and an increased hoof angle. Therefore, the methods according to the present invention can be used to treat, prevent or reduce the severity of the increased tone of the deep and/or superficial digital flexor muscles in a quadruped animal.

The methods according to the present invention are highly suitable for treating, preventing, or reducing the severity of the pathological contraction of the DDF tendon (DDFT) and the SDF tendon (SDFT) in a quadruped animal. In one embodiment, the pathological contraction of the DDFT and the SDFT in a quadruped animal is treated by administering an effective amount of a botulinum toxin, to decrease the tone of the DDF muscle and SDF muscle, respectively. This can reduce tensile forces on the second and third phalanx and result in a reduction of the pain/contraction cycle and normalization of joint angles. Muscles that can be targeted to treat a DDF or SDF abnormality or injury include one or more of the following: deep digital flexor muscle, superficial digital flexor muscle, flexor digitorum profundus, flexor digitorum superficialis, flexor carpi ulnaris, the biceps brachii, triceps, and the serratus ventralis.

The DDFT and the superficial digital flexor are attached to the solar aspect of the coffin bone and the palmar aspect of the second phalanx, respectively. The muscle bellies (flexor digitorum profundus and flexor digitorum superficialis) flex the limb during movement. When a pathological condition arises, growth of the affected hoof starts to become more cylindrical in shape from lack of normal weight distribution and contact with the ground. As the case advances, the angle of the dorsal hoof wall increases, approaching 90 degrees; the heels do not wear, the toe wears excessively and bruising may be evident at the toe. An increased pastern angle may also be noted either in isolation or in combination with the changes of the distal phalanx. These changes can be due to increased tone of the deep and or superficial digital flexor muscle. Therefore, the methods of treating, preventing or reducing the flexural abnormality or injury according to the present invention can be used to reduce or decrease the tone of the deep and or superficial digital flexor muscle. Decreasing the tone of the deep and or superficial digital flexor muscle can result in decreasing the pathological forces on the coffin bone (3r d phalanx) and the second phalanx.

Superficial Digital Flexor Tendon Injuries.

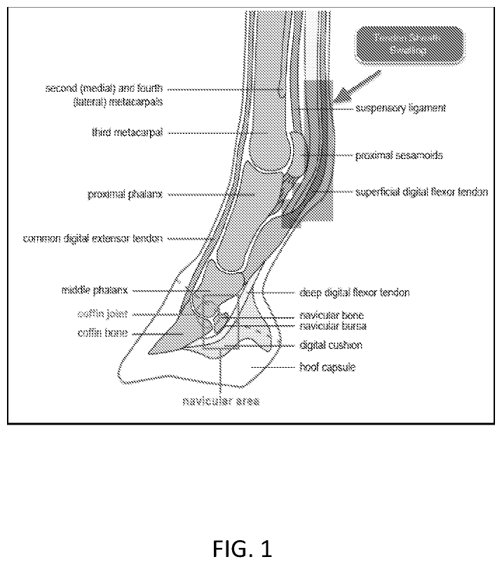

High tensile forces in specific muscle groups of the leg and shoulder can contribute to strain and inflammation of the associated tendon, such as in “bowed tendon” (area of involvement shown in the shaded rectangular area in FIG. 1). Inflammation of the superficial digital flexor tendon can manifest following vigorous exercise with pronounced swelling commonly referred to as a “bowed tendon.” The inflammatory response to injury results in pronounced swelling, heat, pain, and lameness. As such, intervention at the muscular and myoneural level through the administration of botulinum toxin can result in an improvement or stabilization of such pathology. Muscles that can be targeted to treat superficial digital flexor tendon injuries include the superficial digital flexor muscle.

Tendon injury may result from its poor regenerative properties following repetitive loading (strain) and accumulated degeneration of the collagen fibers and collagen-rich extracellular matrix. An inability of the cellular components to maintain and repair the extracellular matrix leads to intrinsic damage and failure. In addition, acute tendon failure may occur when exercise overload exceeds the structural strength of the tendon. Forelimb Superficial Flexor Tendonitis is often bilateral with one limb more severely affected. This can indicate subclinical degeneration of the extracellular matrix prior to overt injury. Additional reasons for an increased susceptibility to injury include the superficial location of the tendon, poor vascular supply (notable in mid metacarpal region), and its association with synovial structures.

Deep and Superficial Muscle/Tendon Contracture.

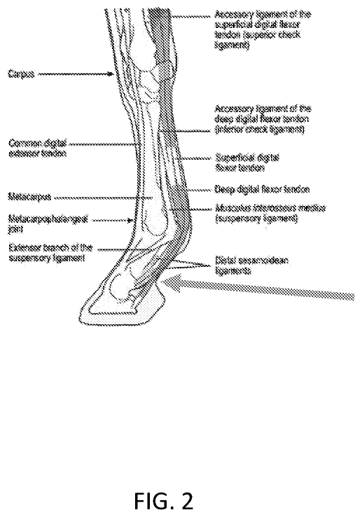

The pathological contraction of the deep digital flexor tendon and the superficial digital flexor tendon can be treated by utilizing botulinum toxin to decrease the tone of these muscles. This can reduce tensile forces on the second and third phalanx and result in a reduction of the pain/contraction cycle and normalization of joint angles. The flexor contracture is a devastating developmental impairment in horses. By using botulinum toxin in horses with this progressive deformity, permanent contracture and loss of economic value can be prevented. The arrow shown in FIG. 2 is directed to the diseased area that will be affected by a botulinum toxin injection. Muscles that can be targeted to treat superficial digital flexor tendon injuries include, but are not limited to, the deep and superficial digital muscles.

The deep digital flexor tendon and the superficial digital flexor are attached to the solar aspect of the coffin bone and the palmar aspect of the second phalanx respectively. The muscle bellies (flexor digitorum profundus and flexor digitorum superficialis) flex the limb during movement. When a pathological condition arises, growth of the affected hoof starts to become more cylindrical in shape from lack of normal weight distribution and contact with the ground. As the case advances, the angle of the dorsal hoof wall increases, approaching 90 degrees; the heels do not wear, the toe wears excessively, and bruising may be evident at the toe. An increased pastern angle may also be noted either in isolation or in combination with the changes of the distal phalanx. These changes are due to increased tone of the deep and or superficial digital flexor muscle and injection of botulinum toxin.

Navicular Syndrome

Navicular syndrome is one of the most common causes of lameness in horses. This condition occurs especially in performance horses, particularly middle age to older animals. Navicular syndrome consists of several clinical signs, including but not limited to pain in the palmar region of the hoof (heel area) and gait abnormalities associated with degenerative changes in the navicular bone (distal sesamoid bone). The degenerative process affects both the bone and the navicular bursa surrounding the bone, as well as the deep digital flexor tendon that runs behind the bursa. This process more often occurs in the forefeet but can affect the hind feet in some cases. Navicular syndrome is usually a bilateral disease (affecting both the right and left sides) with one foot often worse than the other.

Compressive forces and stress on the navicular bone have been investigated. The navicular bone provides a constant angle of insertion and asserts the mechanical advantage of the deep digital flexor tendon on the distal phalanx. In a heel-first landing, the coffin joint rotates decreasing tension in the deep digital flexor tendon. Toe-first impact rotates the coffin joint in the opposite direction as the heels make subsequent downward motion to ground contact, thereby increasing tension on the deep digital flexor tendon and compressive forces upon the fibrocartilage of the navicular bone. Comparisons of normal and presumed navicular syndrome horses have demonstrated that, although mean peak force and stress were similar, the peak forces upon the navicular bone in navicular syndrome horses were approximately double in the early stance phase of stride. It is noted that increased tone of the deep digital flexor muscle results in a toe-first landing which is often noted in the gait of navicular syndrome horses.

In one example of navicular syndrome, there can be an increased or abnormal force on the navicular bone has increased the amount of bone remodeling. This force could be created between the navicular bone and the deep digital flexor tendon. Increased bone remodeling can lead to both pain and lameness.

One method of improving comfort in horses with navicular syndrome is to inject the deep digital flexor muscle with botulinum toxin at the level of the antebrachium to relieve muscle contractility. Muscle atony can cause a decrease in peak pressure of the deep digital flexor tendon upon the navicular bone (distal sesamoid), reducing the pain and slowing progression of fibrocartilage destruction of the navicular bone and deep digital flexor tendon. In addition, subcutaneous injection of botulinum toxin immediately proximal to the coronet and heel bulbs can have uptake and trafficking by sensory neurons to exert a local block of release of neurotransmitters. Evidence suggests a lesser role of tissue inflammation and increased mechanical and neuropathic role in the etiology of navicular syndrome. Botulinum toxin can be used in treating the pathology of sensory endings, fibers and the central nervous system sensitization secondary to chronic degeneration and/or inflammation of the structures necessary for ambulation and proprioception.

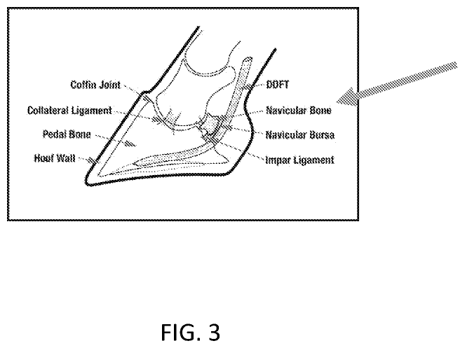

Botulinum toxin is a more economical, less invasive method of resolving the neuropathy causing pain and lameness than the surgeries now being performed to treat the condition of navicular syndrome of the fore and hind limb. Muscles that can be targeted to treat navicular syndrome include, but are not limited to, the deep digital flexor muscle. The disease area affected by the botulinum toxin is shown in FIG. 3.

Suspensory Ligament

The suspensory ligament, along with the sesamoid bones and distal sesamoidean ligaments, functions to prevent excessive extension of the fetlock joint during weight-bearing and the stance phase. Together these structures form a critical component of the fore limb stay apparatus and the hind limb reciprocal apparatus allowing the horse to stand at rest with virtually no muscular effort. Inflammation or injuries to the suspensory ligament can result in a change to a horse's, or other quadruped's, ability to stand, walk, or run.

One example of a suspensory ligament inflammation or injury is suspensory desmitis. Suspensory desmitis is damage and inflammation of the suspensory ligament or its branches caused by excessive loading forces along with fatigue of the limb muscles during intense exercise. There are three anatomical regions of the suspensory ligament: proximal, body, and distal branches. Additionally, degenerative disorders of collagen and pituitary pars intermedia hyperplasia may exacerbate desmitis. Recent studies suggest that pain of chronic suspensory desmitis may have a neuropathic component contributing to disability. The methods according to the invention described herein can be used to treat suspensory desmitis in a quadruped animal by administering or injecting botulinum toxin to the limb of the animal.

Inflammation of the proximal aspect of the interosseus medius muscle, or suspensory ligament (i.e., proximal suspensory desmitis, PSD) of the hind limb, is a common cause of acute or chronic lameness of horses and is most commonly diagnosed in competition horses 4 to 10 years old. The proximal portion of the ligament contains a variable amount of striated muscle fibers and fat tissue that varies by age and breed. The methods of treating, preventing or reducing the suspensory ligament abnormality or injury according to the present invention can be used to treat, prevent, or reduce the severity of the inflammation of the suspensory ligament.

In an embodiment, a method of resolving proximal suspensory desmitis of a hind limb is to inject the suspensory ligament with botulinum toxin to relieve muscle contractility. Muscle atony can cause a decrease in ligament size to resolve entrapment neuropathy and can be a more economical, less invasive method of resolving the neuropathy causing pain and lameness than the surgeries currently being performed to treat the condition of proximal suspensory desmitis of the hind limb. Temporary chemical de-nervation of suspensory ligament muscle fibers can reduce the compressive damage to the deep branch of the lateral plantar nerve associated with lameness caused by proximal suspensory desmitis.

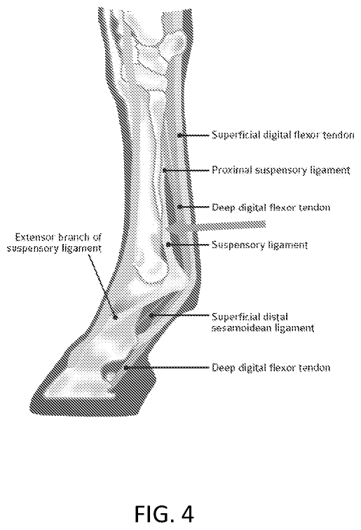

Muscles or ligaments that can be targeted to treat suspensory ligament inflammation or injury include one, but is not limit to, the suspensory ligament or a muscle to which the suspensory ligament is attached. FIG. 4 shows injection sites and disease area affected by the botulinum toxin.

Botulinum Toxin

The botulinum toxin is a neurotoxic protein that prevents the release of acetylcholine from the nerve terminal by disrupting the release mechanism of the acetylcholine-containing vesicles. Blocking the release of acetylcholine from the nerve terminal temporarily prevents the contraction of a muscle, thereby decreasing the tone of the muscle. Through such action, with methods described herein, the balance of forces on the limb or shoulder can be altered, thereby treating the superficial digital flexor tendon injuries, deep and superficial muscle/tendon contracture, navicular syndrome, and suspensory ligament inflammation and injuries. Other mechanisms of action of botulinum toxin are included.

Botulinum toxin consists of seven distinct serotypes, A through G. The present invention should not be limited to any one serotype of the toxin. However, in one embodiment, the botulinum toxin type A is administered to the animal. Type A is commercially available and includes, but is not limited to, Botox®, Allergan, Irvine, California; Dysport®, Ipsen, Paris, France; Xeomin®, Merz, Greensboro, North Carolina; Jeuveau®, Evolus, Newport Beach, California; and Neuronox® and Coretox®, Medytox, Inc, South Korea. In another embodiment, Type B is also commercially available and can be obtained as Myobloc®, Solstice Neurosciences, San Francisco, California.

Typically, the physiologic effect of botulinum toxin lasts 10-12 weeks. Whether multiple treatment regimens (or sessions) are required can depend on the degree of success of the previous treatment. In some embodiments, a quadruped animal will undergo a single treatment. In other embodiments, a quadruped can have 2, 3, 4, 5 6, 7, 8, 9, or 10 treatment regimens. In some embodiments, the time period between treatment regimens can be 1, 2, 3, 4, 5, 6, 7, 8, 9, 10, 11, 12, 13, 14, 15, or 16 weeks.

An effective amount of the botulinum toxin refers to the quantity (amount) that induces a desired response in the animal subject after administration. The total effective amount per limb can vary depending on the type of botulinum toxin, the brand of botulinum toxin, the size of the quadruped, and the size and involvement of the affected muscles in the limb. Optimally, an effective amount produces a therapeutic effect in the absence of, or with little or virtually no, adverse effects or toxicity in the animal. Alternatively, any adverse effects associated with an effective amount are optimally outweighed by the therapeutic benefit achieved. In one embodiment, the total effective amount intramuscularly injected per limb can be an amount equivalent to between about 1 U and about 600 U of Botox® botulinum toxin Type A, such as an amount equivalent to between about 20 U and about 400 U of Botox® botulinum toxin Type A per limb, between about 40 U to 200 U of Botox® botulinum toxin Type A per limb, between about 50 U and 100 U of Botox® botulinum toxin Type A per limb, or between about 100 U and 200 U of Botox® botulinum toxin Type A per limb.

Alternatively, the botulinum toxin, can be administered in an amount equivalent to between about 10⁻³ U and about 60 U of Botox® botulinum toxin type A per kg of the animal per limb to be treated. Preferably, the botulinum toxin used is administered in an amount equivalent to between about 10⁻² U and about 50 U of Botox® botulinum toxin type A per kg per limb to be treated. More preferably, the botulinum toxin is administered in an amount equivalent to between about 10⁻¹ U and about 40 U of Botox® botulinum toxin type A per kg per limb to be treated. Most preferably, the botulinum toxin is administered in an amount of between about 1 U of Botox® botulinum toxin type A per kg and about 30 U/kg per limb to be treated. In a particularly preferred embodiment of the present disclosed methods, the botulinum toxin is administered in an amount equivalent to between about 1 U and about 20 U of Botox® botulinum toxin type A per kg per limb to be treated. It is understood that other brands and/or serotypes of botulinum toxin can have different potency and the dosage can be adjusted accordingly.

As will be appreciated by those having skill in the art, the specific dose can be calculated in the light of the relevant circumstances, including the approximate weight or volume of the muscle targeted for injection, the severity of the tension of the targeted muscle, the age of the animal, and response of the individual animal receiving treatment. Exact dosages can be determined based on standard dose-response studies. Therapeutically effective doses for treatment of afflicted animals can be determined by titrating the amount of the active product given to the animal to arrive at the desired therapeutic effect, while minimizing side effects.

The composition effective for treating flexural abnormalities and injuries, navicular syndrome and suspensory ligament abnormalities and injuries according to the invention comprises botulinum toxin. More particularly, the composition comprises botulinum toxin, e.g., type A, and a pharmaceutical carrier suitable for injection. Typically, the botulinum toxin is administered after being reconstituted according to the manufacturer's recommendations at a desired dilution. In one example, a quantity of 100 U of powdered Botox is dissolved in 2-4 mL of preservative free, sterile saline. The region to be injected is prepped in a sterile fashion. The target muscle is first identified anatomically; the muscle can then be accessed utilizing, for example, an Ambu Neuroline Inoject 24 gauge, 2-3 inch coated needle electrode using EMG guidance. Botulinum toxin is then injected through this needle into one or more sites in the target muscle. For example, botulinum toxin can be injected into 1, 2, 3, 4, 5, 6, 7, 8, 9, 10, or more sites in the target muscle. The injection sites can be spaced apart across the length and width of the muscle. In some embodiments, the spacing between injection sites for a target muscle can be 1, 2, 3, 4, or 5 inches. The dosage per injection site can be an amount bioequivalent to 0.5 U, 1 U, 2 U, 3 U, 4 U, 5 U, 6 U, 7 U, 8 U, 9 U, or 10 U of Botox® botulinum toxin type A. In some embodiments, a target muscle can have 2 to 6 injection sites and the dosage per injection can be an amount bioequivalent to 2 U to 8 U of Botox® botulinum toxin type A. In a treatment cycle, a single muscle can be targeted or a plurality of muscles, e.g., 2, 3, 4, 5, or 6 muscles) can be targeted for botulinum intervention. The total dosage is the total amount that is injected per limb in a treatment session.

The methods of the invention further embrace the administration of pharmaceutically acceptable formulations of the botulinum toxin composition either alone or in combination with other agents or therapies. For example, physical therapy may be utilized in combination with the administration of botulinum toxin. Other therapies which have demonstrated a benefit and are suitable for use with botulinum include hoof farriery, swimming, corrective shoeing, proper trimming, and control of exercise. In some aspects, medical management can be used to control factors contributing to the positive feedback cycle of pain and contraction; restriction of exercise can be used to reduce concussion of growing bone and cartilage, balanced nutrition can be used to normalize bone and joint development and anti-inflammatory medication can be used to reduce pain.

In some aspects, the combination therapy is a second therapeutic such as an anti-inflammatory drug. For example, the anti-inflammatory drug can be a nonsteroidal anti-inflammatory drug (NSAID). Nonsteroidal anti-inflammatory drugs (NSAIDs) are frequently used for their analgesia, antiinflammatory, and platelet aggregation inhibitory effects. Phenylbutazone is the antiinflammatory drug of choice for navicular syndrome. The primary goal of treatment with antiinflammatory drugs is to use the minimum dose possible to maintain maximum comfort and to reduce inflammation, while avoiding adverse toxicity such as gastrointestinal ulceration. The recommended dosage is 2.2 mg/kg twice a day. The lowest dose and shortest duration of treatment is recommended. Sometimes, horses can be effectively managed by giving the NSAIDs the day before and day of competition or riding. Intrarticular (injected into the joint space) corticosteroids, alone or in combination with hyaluronic acid, may provide temporary relief to inflamed structures relating to navicular disease. The corticosteroids of choice are triamcinolone and methylprednisolone acetate. These can be injected into the coffin joint or navicular bursa. Polysulfated glycosaminoglycans (Adequan), isoxsuprine hydrochloride (peripheral vasodilator), and pentoxifylline (hemorreologic agent) are additional treatment options that can be used.

### EXAMPLES

### Example 1—Flexural Deformity

This is a 3 month old walking horse colt that was noted to develop an increased pastern angle bilaterally. The colt is otherwise healthy with no radiographic evidence of developmental orthopedic disease. The colt began to exhibit changes in the shape of his hoof including the development of a more cylindrical hoof.

Utilizing the technique described below, the colt received onobotulinum, 100 units divided into the bilateral deep and superficial digital flexor muscles. This was administered in 12.5 unit aliquots, 2 injections in each of the bilateral superficial and deep digital flexor.

The horse tolerated the procedure well with no adverse effects noted.

The horse was monitored daily showing no signs of adverse effects. The fore feet were trimmed every 2 weeks from 2 months to 6 months of age. From 6 months to one year of age the feet were trimmed monthly. Nutrition and exercise were controlled. Over the next several months there was a slow improvement in the conformation of the forelimbs. At one year of age the horse has visibly normal pastern and foot conformation.

Materials and Methods

Horses at varying stages of orthopedic deformity were identified and evaluated.

Botulinum toxin Type A (Botox®) was diluted with preservative free normal saline to 100 units per 2 ml to assure adequate distribution throughout the target musculature. The region to be injected was prepped utilizing sterile technique.

The Flexor Digitorum Profundus (FDP) injection sites are identified by palpation of the medial aspect of the radius approximately 8 cm distal to the point of the olecrenon, then the belly of the flexor carpi ulnaris, and the vascular bundle posteriorly. The needle is then inserted in the groove between the flexor carpi ulnaris and the flexor digitorum superficialis, avoiding the adjacent neurovascular bundle. The needle is advanced to the radius then backed out approximately 1 cm. A total of 4-8 injections are administered along the axis of the FDP. The superficial flexor is palpated medial to the neurovascular bundle on the posterior aspect of the leg. The needle is advanced approximately 1-2 cm prior to injection of 2-4 sites along the length of the muscle.

An Ambu Neuroline Inoject (Ambu Inc, Glen Burnie MD) 24 gauge, 3 inch coated needle electrode was utilized with electromyography (EMG) guidance. The total amount of Botulinum Toxin Type A intramuscularly injected ranged from 100 units to 200 units per limb depending on the size of the horse and the involvement of the affected muscles.

### Example 2—Flexural Deformity

This is a 4 month old thoroughbred filly who developed an increasing pastern angle bilaterally. She was also noted to exhibit hyperextension of the left fore carpus. The horse was otherwise healthy with no radiographic evidence of underlying hoof or leg disease. Utilizing the above technique described in Example 1, the filly received a total of 200 units Botox. The left fore leg received 100 units divided as 3 injections of 25 units each into the superficial flexor, and 3 injections of 25 units into the deep flexor. An additional 50 units was injected into the right deep digital flexor as 2 injections of 25 units each. Two weeks later, after ensuring tolerability, an additional 50 units was administered as two 25 unit aliquots into the deep digital flexor of the left forelimb.

Feet were trimmed every 3 weeks from 4-8 months of age. At 2 months post injection she showed normal pastern angle, hoof conformation, and hyperextension at the knee had resolved. 4 months after injection she remained normal.

### Example 3—Flexural Deformity

A 6 month old thoroughbred was noted on initial examination to have an increased hoof angle in the left forelimb. The horse was otherwise healthy with no evidence of underlying metabolic disease or limb anomaly. The horse was initially treated with a regimen of hoof trimming every 2 weeks, and a controlled exercise and diet regimen. He failed to respond to this conservative regimen.

Utilizing the technique described in Example 1, at 13 months of age the colt received 200 units of botulinum toxin type A into the deep digital flexor muscle of the involved limb.

The horse was monitored with no adverse effect noted. Examination at 1 month post showed stabilization of the limb.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

What is claimed is:

1. A method for treating superficial digital flexor tendon injuries, deep and superficial muscle/tendon contracture, navicular syndrome, and suspensory ligament inflammation and injuries of a quadruped animal comprising: injecting a botulinum toxin, in a therapeutically effective amount, into one or more tissues or muscles of the animal.

2. A method for treating Navicular syndrome in the limb of a quadruped animal comprising:
injecting a Botulinum Toxin, in a therapeutically effective amount, into one or more muscles of the limb or shoulder of the animal,
wherein the one or more muscles, tendons, or ligaments of the limb comprises or consists of the deep digital flexor muscle.

3. A method for treating suspensory ligament inflammation or injury in the limb of a quadruped animal comprising:
injecting a Botulinum Toxin, in a therapeutically effective amount, into one or more muscles, tendons, or ligaments of the limb or shoulder of the animal,
wherein the one or more muscles, tendons, or ligaments of the limb comprises or consists of the suspensory ligament or the attached thereto.

4. A method for treating a deep and/or superficial muscle/tendon contracture in the limb of a quadruped animal comprising:
injecting a Botulinum Toxin, in a therapeutically effective amount, into one or more muscles of the limb of the animal,
wherein the one or more muscles, tendons, or ligaments of the limb comprises or consists of the deep digital flexor muscle, superficial digital flexor muscle, flexor digitorum profundus, flexor digitorum superficialis, flexor carpi ulnaris, the biceps brachii, triceps, and the serratus ventralis.

5. A method for treating superficial digital flexor injury or abnormality in the limb of a quadruped animal comprising:
injecting a Botulinum Toxin, in a therapeutically effective amount, into one or more muscles of the limb of the animal,
wherein the one or more muscles, tendons, or ligaments of the limb comprises or consists of the superficial digital flexor muscle.

6. The method of any one of claims 1 to 5, wherein the botulinum toxin is botulinum toxin type A.

7. The method of any one of claims 1 to 6, wherein the botulinum toxin is injected into one or more muscles further comprising a forelimb synsarcosis, wherein the one or more muscles of the forelimb synsarcosis are selected from brachiocephalicus, omotransversarius, trapezius, rhomboideus, cutaneous trunci, ascending pectoral muscles, descending pectoral muscles, transverse pectoral muscles, and latissimus dorsi.

8. The method of any one of claims 1-7, wherein at least 30% of a total dosage per limb of the botulinum toxin is injected into the one or more muscles of the limb of the animal.

9. The method of any one of claims 1 to 8, wherein a total dosage per limb of the Botulinum Toxin is an amount of Toxin bioequivalent to an amount between about 40 Units to 200 Units of Botox®.

10. The method of any one of claims 1 to 9, wherein the quadruped animal is a horse.

11. The method of any one of claims 1 to 10, wherein the quadruped animal is a filly or a colt or less than 3 years old or wherein the quadruped animal is a foal or less than a year old.

12. The method of any one of claims 1 to 11, wherein the quadruped animal is an adult or greater than 3 years old.

13. The method of any one of claims 1 to 12, wherein the quadruped animal is a hooved animal and exhibits an abnormally low pastern angle.

14. The method of any one of claims 1 to 13, wherein the quadruped animal is a hooved animal and exhibits an abnormally high pastern angle.

15. The method of any one of claims 1 to 14, further comprising administering a second therapy to the quadruped animal.

16. The method of claim 15, wherein the second therapy is an anti-inflammatory therapeutic.

17. The method of claim 16, wherein the anti-inflammatory therapeutic can be administered intravenously, intraperitoneally, or subcutaneously.

18. The method of claim 15, wherein the second therapy is hoof trimming, controlled exercise, or a diet regimen.

19. The method of any one of claims 1-18, further comprising injecting a second Botulinum Toxin, in a therapeutically effective amount, into one or more muscles of the limb or shoulder of the animal.

20. The method of 19, wherein each injection is half of a total dosage per limb.

21. The method of 19, wherein each injection is a total dosage per limb.

22. The method of any one of claims 4-21 wherein the hoof and pastern angle are improved.

23. The method of any one of claims 4-21, wherein the knee of the animal returns to a position found in healthy animals.

24. The method of any one of claims 2, 3, and 6-21, wherein muscle contractility is relieved.
